(19) United States
(12) Patent Application Publication
(10) Pub. No.: US 2009/0157031 A1
(43) Pub. Date: Jun. 18, 2009

(54) Absorbent article comprising a containment flap having an elastic member and a resilient member

(76) Inventors: Yung H. Huang, Appleton, WI (US); Russell J. Brumm, Menasha, WI (US); Sara Stabelfeldt, Appleton, WI (US); Paul T. Van Gompel, Hortonville, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/001,868

(22) Filed: Dec. 13, 2007

(51) Int. Cl. A61F 13/45; B23P 11/00

(52) U.S. Cl. 604/369; 604/385.24; 29/428; 156/163

(57) ABSTRACT

Containment flaps comprising an elastic member and a resilient member are disclosed. When employed in a disposable absorbent article, the resilient member is adapted to be disposed between the elastic member and the body surface of the wearer of the disposable absorbent article. The resilient member serves to help make the containment flaps more comfortable for the wearer, and can help the flap better conform to contours associated with the body surfaces with which the containment flap comes into contact.

### BACKGROUND

People rely on disposable absorbent products in their everyday lives. Many manufacturers seek to better meet the needs of users of such products.

Product developers seek to design disposable absorbent products that do not leak. For this reason, some product developers employ containment flaps in the design of a disposable absorbent product. Typically containment flaps are placed on either side of the crotch region of the disposable absorbent article. Such flaps often include a thin, flexible substrate (i.e., a flap web) attached to, and protruding from, the body-facing surface of the crotch region. An elastic member attached to this flap web—typically at a location some distance away from the area of attachment between the flap web and the body-facing surface of the crotch region of the article—helps hold some portion of the flap web against the skin of the user of the article. The result can be something like a U-shaped bucket when a cross-section of the article is viewed from the front. That is, each containment flap may be thought of as something like a “wall”—or the sides of the letter “U”—on either side of the crotch region of the disposable absorbent article, with this crotch region forming the bottom of the letter “U.” Ideally, body fluid released from a user (e.g., urine) is contained within this “U”, with some or all of this fluid being absorbed into an absorbent core. This means that the flap ideally forms something like a gasket against the skin of the wearer.

One product-design issue associated with containment flaps having an elastic member is comfort. The elastic member may press against the skin such that the user experiences discomfort, sometimes to the extent that red marks are left on the skin when a user, or caregiver, removes the article. Generally the elastic member does not contact the skin directly. Instead the elastic member presses against the flap web—which is disposed between the elastic member and the skin—and this portion of the flap web is then pressed against the skin by the elastic member. Also, the elastic member is typically not capable of expanding to fill any depressions in skin or corresponding tissue. In other words, elastic members generally are not adapted to conform to the potentially complex contours of the body surface with which containment flaps come into contact. So a gasket-like seal between some portion of the containment flap, and the skin of the wearer, may not be formed.

For example, imagine a small-diameter, stretched, elastic strand, attached at one end to the palm of an outstretched hand, and attached at its other end to the bicep on the same arm. When the arm is held out straight, the elastic strand conforms somewhat to the contours of the arm from the bicep to the palm where the strand is attached at its ends, although there will still likely be some gaps between the strand and the underlying skin. Then imagine the arm so that the forearm is at a right angle to the upper arm. If the elastic strand remains in a stretched condition (although less stretched compared to when the arm is in an outstretched condition), it will run straight from the palm of the hand to the bicep, with a large gap between much of the strand and the crook of the arm near the elbow. The strand is not capable of filling this space or gap, or conforming to the more complex surface formed when the forearm is at a right angle to the upper arm.

### SUMMARY

We have conceived novel disposable absorbent articles employing containment-flap designs that should help provide additional comfort to a user of the article. Furthermore, such flap designs may better facilitate the flap conforming to complex contours associated with body surfaces with which the flap typically comes into contact (e.g., depressions in such body surfaces). In effect, the novel flaps, in the figurative context discussed above, could expand to fill, or fill some portion of, the crook in the arm. That is, the flap can expand, thereby possessing gap-filling properties.

We have discovered that a resilient member that is interposed between the elastic member and the human body is able to conform more readily to the contours of a user's body. Furthermore, the material(s) of construction, and the shape, of the resilient member may be selected so that the resilient member is better able to conform to the potentially complex contours of the body surfaces with which the flap typically comes into contact. For example, the resilient member may be adapted to fill or partially fill a depression in the body surface (e.g., as with a resilient member comprising foam). In some versions of the invention, the resilient member is not attached to the elastic member.

We have discovered that the resilient member desirably has certain properties relative to the properties of the elastic member. Specifically, the stress-strain curve of the resilient member compared to the stress-strain curve of the elastic member is such that the relative properties of the elastic member and resilient member: (1) allow for both members to be stretched when attached to a web; and (2) provide for a resilient member that is softer than the elastic member. This will typically mean that the elongations of the resilient member and the elastic member are substantially the same when these members are attached to the flap web. When the composite comprising the flap web, elastic member, and resilient member is allowed to retract, these similar elongations will help ensure that the composite does not buckle such that the manufacturing operation or end-use performance is negatively impacted in a significant way. The resilient member will also typically be softer than the elastic member, and perhaps have a different shape than the elastic member, to help provide increased comfort to a user of the article and/or to minimize total product cost. For example, if the elastic member has a substantially cylindrical cross-section, and is of a relatively small diameter, then a resilient member having a rectangular cross-section might be selected, with the longer dimension of this rectangular cross-section being greater than the diameter of the strand, thereby increasing the area of contact between the skin of a user and the corresponding flap employing the elastic member and resilient member (because, as noted above, the resilient member is interposed between the skin of a user and the elastic member). Furthermore, selecting the relative size of the resilient member and elastic member in this way helps spread out the forces acting on the body of the user when the elastic member and resilient member are in a stretched condition. In addition, the resilient member may only partially extend along the elastic member, to fill only specific gaps along the flap elastic-body interface.

One representative version of the invention is a containment flap comprising a web, wherein the web is attached to itself to form an enclosed volume; an elastic member attached to said web, wherein the elastic member is located within said enclosed volume; and a resilient member attached to said web, wherein the resilient member is located within said enclosed volume, and wherein said resilient member is not attached to the elastic member. For purposes of this application, the term “enclosed volume” means a volume formed by the flap web being attached to itself.

Other representative embodiments include the aforementioned containment flap in which: the elastic member is an elastic strand; the resilient member comprises foam; the resilient member comprises at least one pair of substantially parallel outer surfaces (e.g., as with a shape having a rectangular cross section); and/or the cross-sectional area of the resilient member is greater than the cross-sectional area of the elastic member (for cross sections that are contained in an imaginary plane that is perpendicular to a longitudinal dimension along the length of the elastic member or resilient member).

In another representative embodiment, the aforementioned containment flap is employed in a disposable absorbent article.

Another representative embodiment is a containment flap comprising: a web, wherein the web is attached to itself to form an enclosed volume; an elastic member attached to said web, wherein the elastic member is located within said enclosed volume; and a resilient member attached to said web, wherein the resilient member is located outside said enclosed volume, and wherein said resilient member is not attached to the elastic member.

Other representative embodiments include the aforementioned containment flap in which: the elastic member is an elastic strand; the resilient member comprises foam; the resilient member comprises at least one pair of substantially parallel outer surfaces (e.g., as with a shape having a rectangular cross section); and/or the cross-sectional area of the resilient member is greater than the cross-sectional area of the elastic member (for cross sections that are contained in an imaginary plane that is perpendicular to a longitudinal dimension along the length of the elastic member or resilient member).

In another representative embodiment, the aforementioned containment flap is employed in a disposable absorbent article.

Another representative version is a containment flap comprising: a first web; a second web attached to said first web; an elastic member disposed between and attached to at least a portion of the first web, the second web, or both; and a resilient member disposed between and attached to at least a portion of the first web, the second web, or both.

Other representative embodiments include the aforementioned containment flap in which: the elastic member is an elastic strand; the resilient member comprises foam; the resilient member comprises at least one pair of substantially parallel outer surfaces (e.g., as with a shape having a rectangular cross section); and/or the cross-sectional area of the resilient member is greater than the cross-sectional area of the elastic member (for cross sections that are contained in an imaginary plane that is perpendicular to a longitudinal dimension along the length of the elastic member or resilient member).

In another representative embodiment, the aforementioned containment flap is employed in a disposable absorbent article.

Another representative version of the invention is a method of making a containment flap, the method comprising the steps of: providing a flap web; providing an elastic member; providing a resilient member; attaching the elastic member to the flap web, wherein the elastic member, when attached to said flap web, is elongated and characterized by a first elongation; attaching the resilient member to the flap web, wherein the resilient member, when attached to said flap web, is elongated and characterized by a second elongation; allowing the combination of the flap web, elastic member, and resilient member to return to an un-elongated state; wherein the first elongation and the second elongation are similar.

In another representative version of the invention, the aforementioned method is such that the first elongation and the second elongation are sufficiently similar so as to prevent substantial buckling of the combination when allowed to return to an un-elongated state.

In another representative version of the invention, the aforementioned method is such that the elastic member and/or resilient member is attached to the flap web using adhesive.

In another representative version of the invention, the aforementioned method further comprises the step of attaching at least some portion of the resilient member to at least some portion of the elastic member.

In another representative version of the inventive method, the elastic member and the resilient member are attached to the same side of the flap web. In another version of the inventive method, the elastic member and resilient member are attached to opposing sides of the flap web.

### DRAWINGS

FIG. 1 depicts one representative example of a disposable absorbent article employing a containment flap of the present invention.

FIG. 2 depicts one representative example of a disposable absorbent article employing a containment flap of the present invention.

FIGS. 3A, 3B, 3C, and 3D depict representative versions of a containment flap of the present invention.

### DESCRIPTION

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Containment flaps” are generally well known to those skilled in the art. Typical constructions and arrangements for containment flaps are described in U.S. Pat. No. 4,704,116 issued 3 Nov. 1987 to K. Enloe, the disclosure of which is hereby incorporated by reference in a manner consistent herewith. Such constructions may be modified as described herein to employ a resilient member adapted to be interposed between an elastic member, or members, and the skin of a user of an article employing the containment flaps.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as meltblowing processes, spunbonding processes, air laying processes, and bonded carded web processes.

“Stretch” refers to the ability of a material to extend upon application of a biasing force. Percent stretch is the ratio of the amount of stretch or extension following application of a biasing force in a material to the initial dimension of that material. Percent stretch may be expressed as [(stretched length—initial sample length)/initial sample length]×100. For example, if a material having an initial length of one (1) inch is stretched 0.50 inch, that is, to an extended length of 1.50 inches, the material can be said to have a stretch of 50 percent. Alternatively, in the present application, the material can be said to have an elongation of 50 percent.

“Recover” or “recovery” refers to a contraction of a stretched material upon termination of a biasing force following stretching of the material by application of the biasing force. For example, if a material having a relaxed, unbiased length of one (1) inch is elongated 50 percent by stretching to a length of one and one half (1.5) inches the material would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

These terms may be defined with additional language elsewhere in the specification.

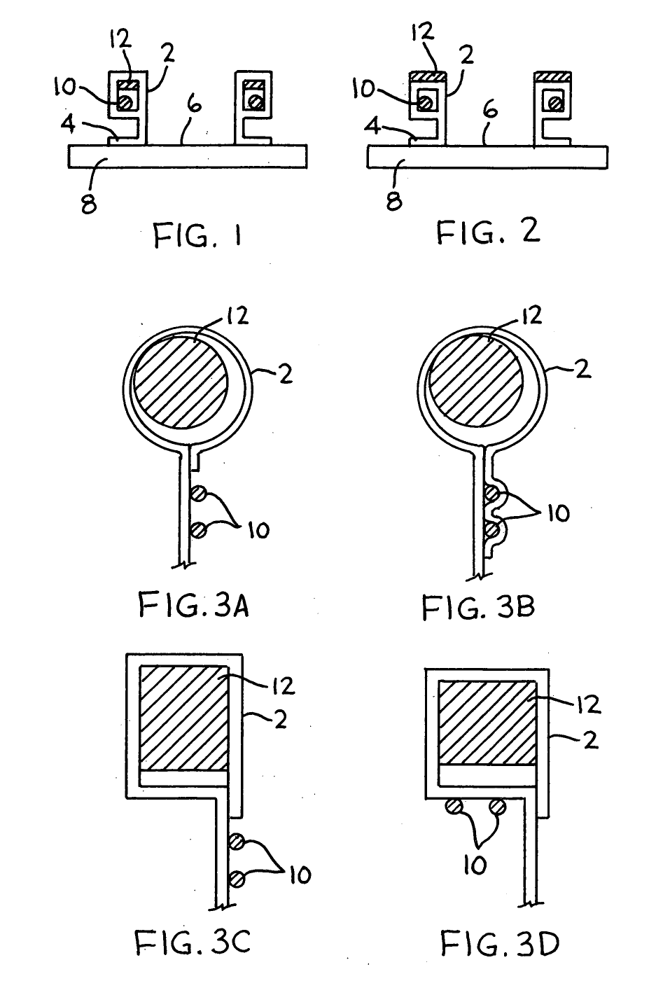

FIG. 1 shows one representative embodiment of the present invention. One portion of a flap web 2, typically an edge portion 4 of the flap web 2, is attached to the body-facing surface 6 of the chassis 8 of a disposable absorbent article (or some component of the chassis, such as another web, during manufacture of the corresponding article). In FIG. 1, a simplified, cross-sectional view of the crotch region of a disposable absorbent is depicted. The flap web may be attached to the chassis, or component of the chassis, using adhesive, stitching, ultrasonic energy, thermal energy, or other such ways of attaching one web or substrate to another. The remaining portion of the flap web—i.e. that portion of the flap web not attached to the body-facing surface of the chassis—protrudes away from the body-facing surface of the chassis. One or more elastic members 10 are typically attached to the flap web to help provide elastomeric qualities to the combination of the flap web and elastic member(s). Furthermore, a resilient member 12 is also attached to the flap web, but in the depicted version not to the elastic member(s), in such a way that the resilient member is adapted to be interposed between the elastic member(s) and the body of a user of the absorbent article when the absorbent article is worn.

Typically the elastic member, when attached to the flap web, is attached in a stretched state. That is, the elastic member, such as an elastic strand, is stretched before it is attached to a surface of the flap web. Typically an adhesive is applied to the strand and/or flap-web surface before, or concurrent to, attachment of the strand to the flap-web surface. Often the adhesive is applied intermittently along the length of the elastic member and/or flap web surface so that those portions of the elastic member between points of attachment can retract when tension on the elastic member is relaxed. When the combination of the elastic member attached to said flap web is allowed to retract, the resulting composite (i.e., the combination of the flap web and the elastic member) is typically gathered.

Accordingly, when making the inventive containment flap, the resilient member should be attached to the flap web so that, when the elastic member retracts, the resilient member retracts in a similar fashion. Otherwise the containment flap will buckle or deform in some manner. For example, assume that the elastic member is attached to the web in a stretched state, and the resilient member is attached in a completely unstretched state. When the combination of the flap web, stretched elastic member attached to said web, and unstretched resilient member attached to said web, is allowed to retract, the elastic member will seek to return to a relaxed, untensioned state. But in so doing, retraction of the elastic member will, in effect, create compressive forces on the already relaxed resilient member, thereby causing it to bend or curve in some manner. The result: a buckled composite (i.e., the combination of the flap web, elastic member, and resilient member) that may look unsightly to the consumer; may be difficult to handle in subsequent manufacturing operations prior to final assembly of the associated disposable absorbent article; may be difficult to package; and/or may not function properly as a containment flap when the article is used, that is, it may not properly fill the gaps that occur between the flap elastic and the body part that is convex or becomes convex upon movement (such as that area at the surface interface of the thigh and the torso in the crotch).

Alternatively, assume that when the elastic member is attached to the web in a stretched state, the resilient member is elongated significantly more than the elastic member. When the elastic member is allowed to retract to its untensioned state, the resilient member will continue to retract—having been initially elongated much more than the elastic member, again resulting in a buckled composite having one or more problems like those identified above.

To avoid these potential problems, both the elastic member and the resilient member should desirably be at approximately the same state of elongation when attached to the flap web. When the resulting flap composite is allowed to retract, the composite should not unduly buckle (i.e., the composite should not assume some gnarled shape that poses one or more problems like those identified above).

The resilient member and the elastic member may be attached to the flap web in various ways, so long as when the absorbent article comprising the flap composite is worn, the resilient member is interposed between the elastic member and the body. So, for example, as depicted in FIG. 1, the resilient member and the elastic member can both be attached to the same surface of the flap web. For the representative embodiment depicted in FIG. 1, the flap web is attached to itself so that the elastic member and resilient member are both positioned within a volume defined by the attachment (i.e., by attaching the flap web to itself an opening analogous to the eye of a needle or closed hook is formed, with the elastic member and resilient member positioned within this “eye”). Alternatively, as depicted in FIG. 2, the resilient member and the elastic member can each be attached to opposing surfaces of the flap web. When the flap web is attached to itself as described for FIG. 1, the elastic member is positioned within the volume and the resilient member is positioned outside the volume. It should be noted that the length of the resilient member may be the same as, more than, or less than the length of the elastic member in the finished flap and article. Furthermore, converting operations may be employed so that the ends of the resilient member are tapered—that is have a reduced thickness—at a location proximate to where the ends of the containment flaps are attached to the chassis.

Note that the flap web need not be attached to itself as described above. Instead two separate flap webs may be used with the elastic member and resilient member sandwiched between these webs. Thus both the elastic member and resilient member may be elongated prior to attachment between the flap webs, with the resulting flap composite being allowed to retract after said attachment. As noted above, the resilient member would be positioned relative to the elastic member so that the resilient member was interposed between the elastic member and the body of the wearer of the corresponding garment.

Other representative versions of containment flaps of the present invention are depicted in FIGS. 3A, 3B, 3C, and 3D. The numeric designators correspond to the same basic components identified in FIGS. 1 and 2 above. Other versions are possible, so long as, in the disposable article employing inventive flaps of the present invention, the resilient member is disposed between the elastic member and the body of the user when the containment flap is deployed (i.e., when the corresponding disposable absorbent article to which the flap is attached is worn).

The containment flaps of the present invention may be employed in a wide variety of disposable absorbent articles, including diapers, training pants, adult-incontinence articles, absorbent pads, and other such articles used to help contain bodily fluids.

The materials of construction of the flap web or web will typically include fibrous substrates, films, or both (depending on whether a product designer wishes the flaps to be permeable or impermeable to the passage of liquids and any associated solids, dissolved or otherwise, through the corresponding containment flap). Frequently the fibrous substrate will be a nonwoven material, such as a spunbond or meltblown web. Also, the flap web itself may have one layer or multiple layers. For example, a flap web may be a film sandwiched between two nonwoven layers (e.g., a film with one spunbond layer attached to one surface of the film, and a second spunbond layer attached to the opposing surface on the other side of the web). If a flap web is a multilayer web, the layers may be attached using a variety of methods and materials. Often the manner in which these layers are attached to one another will be similar to the methods and materials used when attaching an elastic member or resilient member to a flap web including, for example, use of adhesive, stitching, ultrasonic energy, thermal energy, or other such ways of attaching one web or substrate to another.

Any material used for making an elastic member may be used with the current invention. Often the elastic member is made using various polymeric materials that exhibit elastomeric properties and include, for example, materials sold under the brand names Lycra, Spandex, and other such elastomeric materials. These materials may comprise different polymer chemistries (e.g., polyether or polyester chains linked to polyurethanes; or other such chemistries known in the area of making elastic materials). Furthermore, the elastic member can be of various shapes. For example, the cross section of an elastic member can be circular, elliptical, rectangular, square, or other such shape. The elastic member can take the form of a strand, ribbon, tube, sheet, film, fibrous web (e.g., meltblown elastic fiber) or other configuration. Typically the diameter of a substantially cylindrical elastic strand is from about 0.2 mm to about 0.3 mm.

The material or materials used for the resilient member should be such that the resilient member may be elongated in a manner similar to the elastic member. One type of material suitable for use as a resilient member is foam. An example of one such foam is foam material used commercially in a make-up sponge, and is available under the designator P-07 A-07 for PJ1135, Tera Sheet 4t from “The Penthouse Group,” a business having offices in Freeport, N.Y.

Also, the resilient member may be fashioned into a shape that helps distribute the tension of the elastic members over a greater area of contact compared to the elastic members alone, thereby enhancing comfort for the wearer of the corresponding disposable absorbent article employing the inventive containment flaps. It should be noted that when the term “area of contact” or “line of contact” is used, the resilient member or elastic member may not be in direct contact with the skin. As noted above, the flap web may be disposed between the resilient member or elastic member and the surface of the user's skin.

For example, if the elastic members are of a cylindrical shape with a certain radius, the resilient member may be fashioned into a shape having an area of contact that is greater than the area of contact of the elastic members alone. Theoretically, a cylindrical elastic strand would contact (or press flap web material against) the skin roughly along an imaginary line traveling the length of the cylinder (of course this would vary somewhat due to deformation of the cylinder, and due to the propensity of skin and underlying tissue to flex and conform somewhat around some portion of the cylinder). But if a resilient member was interposed between the cylindrical elastic strand and the skin, and the resilient member was shaped so that the area of contact between the resilient member and the skin was larger than the area of contact (or line of contact) between the elastic member and skin alone, then comfort for the wearer of the corresponding article should be increased.

Typically the maximum thickness of an uncompressed resilient member, in a direction generally perpendicular to the body surface with which the flap comes into contact, will be from about 1 millimeter to about 5 millimeters. Suitably this thickness will be from about 3 millimeters to about 4 millimeters.

The cross-sectional shape of the resilient member may be square, rectangular, elliptical, cylindrical, or other such shape. Typically a shape, such as a square or rectangular shape, will be used to increase the area of contact between the resilient member and the body surface.

As noted above, the material with which the resilient member is made will typically be such that the elongations of the resilient member and elastic members are substantially similar to one another, in order to avoid wavy shapes or buckles in the corresponding flap. Such wavy shapes and buckles will form if the resilient member and elastic member have different elongations when attached to the corresponding flap web(s), so that when the flap web composite (i.e., the web and the resilient member and elastic member attached to said web) is allowed to relax, the elastic member and resilient member will seek to retract to different lengths.

### EXAMPLES

### Prophetic Example 1

### Detailing One Representative Embodiment

Two cylindrical, Lycra-brand elastic strands, from Investa, a company having offices in Mississauga, Ontario, are obtained. These strands have a diameter of about 300 to 400 micrometers. A rectangular foam identified as P-07 A-07 for PJ1135, Tera Sheet 4t, is obtained from “The Penthouse Group,” a business having offices in Freeport, N.Y. The foam is 5 millimeters thick.

A web comprising a 3-layer laminate is continuously unwound and directed to a nip between two rolls. The laminate comprises an elastomeric, polypropylene film sandwiched between two polypropylene spunbond facings. Each of the spunbond facings has a basis weight of 0.38 ounces per square yard.

The two reels of elastic strand are continuously unwound at a speed slower than the speed of the web. A hot-melt adhesive, manufactured by Bostik-Findley, a business having offices in Wauwatosa, Wis. is meltblown on to each of the two strands at an add-on level of 0.1 grams per square meter. The relative speed of the elastic strand and the web are such that the strand is elongated by about 250% when attached to the web prior to the combination passing through the aforementioned nip. The strands are positioned inward from the side edge of the web, with the strands being spaced apart by about 80 millimeters.

The resilient member, in this case the foam having a rectangular cross section, is unwound at a speed similar to that of the elastic strand. The same hot-melt adhesive used to attach the elastic strands is applied to the foam at an add-on level of 0.05 grams per meter prior to its attachment to the web. The resilient member is elongated by about the same amount as the elastic strand when attached to the web prior to passing through the aforementioned nip. In this particular prophetic example, the foam is attached to the same side of the web as the elastic strands. The foam is attached at a position between the longitudinal edge of the web, and the elastic strand closest to this longitudinal edge.

After the combination the elastic strand and foam are attached to the flap web, the longitudinal edge portion of the flap web is folded over, and attached to itself so that the foam and elastic strand are within a volume, or “eye,” defined by this attachment. In other words, a containment flap akin to that depicted in FIG. 1 is made, with the exception that this example describes attachment of two elastic strands to the flap web thus resulting in the corresponding containment flap employing two elastic strands (versus the single elastic member depicted in FIG. 1) combination is passed through a nip, the combination is allowed to relax. In this representative embodiment, both the resilient member and the elastic members are positioned within the enclosed volume. In subsequent converting steps, the flap web composite is cut, and attached to other webs or components used in the making of a disposable absorbent article.

We claim:

1. A containment flap comprising:
a flap web, wherein the web is attached to itself to form a enclosed volume;
an elastic member attached to said web, wherein the elastic member is located within said enclosed volume;
a resilient member attached to said web, wherein the resilient member is located within said enclosed volume, and wherein said resilient member is not attached to the elastic member.

2. The containment flap of claim 1 wherein the elastic member is elastic strand.

3. The containment flap of claim 1 wherein the resilient member comprises foam.

4. The containment flap of claim 3 wherein the resilient member comprises at least one pair of substantially parallel outer surfaces.

5. The containment flap of claim 1 wherein the cross-sectional area of the resilient member is greater than the cross-sectional area of the elastic member.

6. A disposable absorbent article comprising the containment flap of claim 1.

7. A containment flap comprising:
a flap web, wherein the web is attached to itself to form a enclosed volume;
an elastic member attached to said web, wherein the elastic member is located within said enclosed volume;
a resilient member attached to said web, wherein the resilient member is located outside said enclosed volume, and wherein said resilient member is not attached to the elastic member.

8. The containment flap of claim 7 wherein the elastic member is elastic strand.

9. The containment flap of claim 7 wherein the resilient member comprises foam.

10. The containment flap of claim 9 wherein the resilient member comprises at least one pair of substantially parallel outer surfaces.

11. The containment flap of claim 7 wherein the cross-sectional area of the resilient member is greater than the cross-sectional area of the elastic member.

12. A disposable absorbent article comprising the containment flap of claim 7.

13. A containment flap comprising:
a first web;
a second web attached to said first web;
an elastic member disposed between and attached to at least a portion of the first web, the second web, or both;
a resilient member disposed between and attached to at least a portion of the first web, the second web, or both.

14. The containment flap of claim 13 wherein the elastic member is elastic strand.

15. The containment flap of claim 13 wherein the resilient member comprises foam.

16. The containment flap of claim 15 wherein the resilient member comprises at least one pair of substantially parallel outer surfaces.

17. The containment flap of claim 13 wherein the cross-sectional area of the resilient member is greater than the cross-sectional area of the elastic member.

18. A disposable absorbent article comprising the containment flap of claim 13.

19. A method of making a containment flap, the method comprising the steps of:
providing a flap web;
providing an elastic member;
providing a resilient member;
attaching the elastic member to the flap web, wherein the elastic member, when attached to said web, is elongated and characterized by a first elongation value;
attaching the resilient member to the flap web, wherein the resilient member, when attached to said web, is elongated and characterized by a second elongation value;
allowing the combination of the web, elastic member, and resilient member to return to a substantially un-elongated state;
wherein the first elongation value and the second elongation value are similar.

20. The method of claim 19 wherein the first elongation value and the second elongation value are sufficiently similar so as to prevent substantial buckling of the combination when allowed to return to a substantially un-elongated state.

21. The method of claim 19 wherein the elastic member is attached to the flap web using adhesive.

22. The method of claim 19 wherein the resilient member is attached to the flap web using adhesive.

23. The method of claim 19 wherein the resilient member and the elastic member are attached to the same side of the flap web.

24. The method of claim 19 wherein the resilient member and the elastic member are attached to opposing sides of the flap web.

25. The method of claim 19 wherein some portion of the resilient member is attached to some portion of the elastic member.
